(19) United States
(12) Patent Application Publication
(10) Pub. No.: US 2026/0358241 A1
(43) Pub. Date: Nov. 12, 2026

(54) MULTI-TRANSAXIAL BIOLOGICAL SAMPLE ANALYSIS SYSTEM AND RELATED METHODS

(71) Applicant: Thread Bioscience, Inc., Allentown, PA (US)

(72) Inventors: Craig SERIO, Bethlehem, PA (US); Brian FELDMAN, Macungie, PA (US)

(73) Assignee: Thread Bioscience, Inc., Allentown, PA (US)

(21) Appl. No.: 19/203,940

(22) Filed: May 9, 2025

(51) Int. Cl. G01N 21/31; G01N 21/3577; G01N 33/493

(52) U.S. Cl. CPC G01N 21/3151; G01N 21/3577; G01N 33/493; G01N 2021/3155; G01N 2021/3181

(57) ABSTRACT

A multi-transaxial spectrophotometer for measurement of biological cell mass and metabolism to determine the presence or absence of log phase bacterial growth in a liquid specimen sample. The apparatus determines the presence or absence of bacterial infection by monitoring changes in green (approximately 560nm) and infrared (approximately 880nm) light transmittance through the sample which is urine of a patient suspected to have a urinary tract infection. Instead of using a single green and IR light path for measurements, multiple green and IR light sources and multiple light sensors transmit light through many different selectable paths which can be independently monitored. This allows measurement of absolute and raw light transmittance values in the sample for enhanced detection accuracy, but in addition light transmittance values from different paths can be compared to detect other potential characteristics or phenomena occurring in the sample.

### BACKGROUND

The present invention relates generally to systems, apparatuses, and methods for analyzing fluid samples, and more particularly to the same for analyzing biological fluid samples for the presence of bacteria.

Urinary tract infections (UTIs) are one of the most common bacterial infections. When a patient presents to a medical provider with symptoms that may be consistent with those of a bacterial infection such as a UTI, a urine sample is typically obtained from the patient for analysis. A diagnosis must be performed to determine if the biological fluid sample has a log phase bacterial growth, or if the patient’s symptoms may be caused by something else.

One common method to test the urine sample for log phase bacterial growth is to send the fluid sample to an outside lab for a culture. The process of sending the fluid sample to a lab and waiting for the result typically takes 2-3 days and the bacterial counts are performed manually. Not only does this process take several days to provide results, but the results can be inaccurate due to the use of urine samples that have gone stale, been contaminated prior to testing, and/or inaccuracies associated with the traditional manual lab methods used to count bacteria. Often antibiotics are preemptively prescribed to a patient before the results of the culture are available without knowing if the patient really has a UTI, which can result in antibiotic resistance. Increasing populations of antibiotic resistant bacterial strains is a continuing concern for health care providers.

Another method used to detect UTIs is PCR (Polymerase Chain Reaction), which also requires a sample to be collected and sent to an outside lab. The main disadvantages of the different forms of PCR tests are also the inconvenience and need for the health care provider to send the collected urine fluid sample to an outside lab, delay in obtaining the test results, and in addition the considerable expense of this test.

Accordingly, there remains a need for improvements in testing of biological fluid samples for UTIs which can improve the timeliness and accuracy of results.

### BRIEF SUMMARY

The present application discloses an automated processor-controlled system and related methods for analyzing a biological fluid specimen or sample such as but not limited to urine to detect and measure bacteria growth in the sample fluid. In one non-limiting embodiment, the system includes an analysis apparatus comprising a multiple transaxial spectrophotometer configured and operable to measure biological cell mass and metabolism to determine the presence or absence of log phase bacterial growth in the fluid sample. The present embodiment specifically relies on monitoring the change of the values over time to detect any changes in the sample that may indicate the presence of bacteria. In one embodiment, the apparatus may be a quad transaxial spectrophotometer in which light signals are both transmitted to and received from the biological sample fluid along four transversely oriented light axes.

The present analysis apparatus in one embodiment generally includes a sample holder including an elongated open well or receptacle which insertably receives a sample container such as an elongated sealed vessel such as an ampoule in one embodiment which holds the biological fluid sample collected from an individual. A plurality of light sources distributed along a first side of the sample holder are operable to illuminate the sample fluid with various wavelengths of light at a plurality of elevations in a direction transverse to the longitudinal axis of the sample holder which extends along the length of the receptacle. Concomitantly, a plurality of light sensors in visual communication with the receptacle and sample container detect and receive the light passing through the sample fluid. The light sensors which may be photodetectors in one embodiment are distributed along a second side of the sample holder to receive the light signals from the illuminate sample in a direction transverse to the longitudinal axis. A programmable microcontroller operably and communicably linked to the light sources and sensors is configured via programming or instructions (e.g., control logic) to control the sample illumination and light detection process. The microcontroller is configured to analyze the biological fluid sample to compare bacterial mass and metabolism measurements obtained from a plurality of preprogrammed testing sequence of illumination and detection cycles conducted over a test period of time to rapidly determine the presence or absence of active log phase bacterial growth in the sample.

The present analysis apparatus determines the presence or absence of bacterial infection by monitoring changes in green light transmittance (approximately 560 nanometers) and infrared (IR) light transmittance (approximately 880 nanometers) through a sample. In the present embodiment, the sample is urine from a patient suspected to have a UTI mixed with a chemical formulation (e.g., reagents or chemical indicators) mentioned in commonly owned U.S. Patents 8,679,775, 8,257,627, and 7,193,717 in order to enable the metabolization of bacteria in the sample to be measured spectrophotometrically. It was previously determined that bacterial growth could be monitored by changes in IR light transmittance and bacterial metabolization could be monitored by changes in green light transmittance. The raw values of the spectrophotometric readings and the changes in readings throughout the test period are used to determine the presence of bacterial growth and bacterial metabolism.

The infectious bacteria that cause UTIs often have a replication cycle of approximately 20 minutes at normal temperatures inside the human body of approximately 37 degrees C (Celsius). Growing and replicating bacteria would reduce the transmittance of IR light. Metabolization by the bacteria would cause a color change in the patented chemical formula added to the sample, which would reduce the transmittance of the green light. Clinical testing confirmed the performance and viability of the basic technology but the enhancements contained in the current invention and disclosure are necessary to use this technology in a marketable product. One of the main enhancements of the current invention is the ability to use shorter (trans-axial) light paths of e.g., 1.2cm for straight trans-axial readings and slightly longer paths for readings taken from a light source at one level/elevation and received by a light detector or sensor (e.g., photodiode) at a higher or lower level/elevation on a generally diagonal path through the sample fluid instead of relying solely on a long light path of e.g., 9cm for the necessary sensitivity. Accordingly, the longer lights paths are advantageously not fixed but variable depending on which light source is used to transmit the light signal into the biological sample fluid and at which elevation the light sensor is selected and located to receive the light signal returned from the sample fluid.

Instead of one green light path and one IR light path, the present spectrophotometric analysis apparatus uses multiple green light sources and multiple IR light sources combined with multiple light sensors (photodiodes) distributed along opposite sides of the sample container to provide for the transmittance of light through many different light paths which are preprogrammed, controlled, and independently monitored by the microcontroller. This allows for the measurement of absolute and raw light transmittance values in and form the sample, as had been done in the past. However, in the present invention, the light transmittance values from different light paths can be compared to each other by the microcontroller to detect other potential characteristics or phenomena occurring in the sample such as for example without limitation such as for example without limitation settling of material in the sample, turbidity, or presence of suspended particles. The use of multiple light paths which are varied over time during the sample test period can also help to ensure that the testing machine is operating properly and that a given light source or light sensor is functioning as desired and does not have degraded performance. If performance of a component has degraded, it is possible to use methods via programmed routines implemented by the microcontroller to account for this degradation to avoid the degradation having an adverse effect on the final measurement thereby safeguarding the accuracy of results.

The microcontroller of the present multi-transaxial spectrophotometric analysis apparatus selectively activates different combinations of light sources and sensors at different times according to a preprogrammed testing sequence during the test period in numerous combinations to create multiple different light signal paths (in length and direction) through the biological sample fluid. This yields more light transmittance measurement data points which are recorded and compared by the microcontroller to in turn advantageously produce more accurate detection results than obtainable by past testing approaches with far fewer measurement data points.

There are multiple other benefits provided by the present multi-transaxial analysis apparatus and related process for spectrophotometric analysis of biological sample fluids in contrast to prior testing approaches.

For example, the present multi-transaxial analysis apparatus is configured and operable to accurately and rapidly determine log phase bacterial growth in a sample, accurately and rapidly determine bacterial metabolism in a sample, minimize disruption in measurements due to effects of external factors such ambient light, minimize disruption in measurements due to turbidity, settling, and other potential sources of disturbance in the sample, and monitor and ensure proper performance of the components of the spectrophotometer.

Advantageously, the present multi-transaxial analysis apparatus also allows the foregoing biological sample fluid tests to be performed at a reasonable cost and at the point-of-collection such as at doctor’s office or in a hospital environment which avoids the need to send samples outside to a lab for preparation of cultures and manual bacterial counts. The present analysis apparatus obviates the need for cultures and rapidly provides results in situ in a matter of 30 minutes or less as opposed to days as used in past approaches. This allows more timely and expedient treatment of patients who can be prescribed an appropriate and accurate course of treatment sooner. Furthermore, the process implemented by the analysis apparatus using a spectrophotometric method with multiple sample illumination and detection points provided improved accuracy of the test results that surpasses manual bacterial count approaches of the past.

In addition, some spectrophotometry machines are calibrated using ampoules filled with special chemical formulations to provide absolute light transmittance readings at different values. Due to the method of ampoule production, the ampoules have much more variation in geometry along the long light paths previously used such as shoulder height, shoulder angle, and thickness of the glass at the shoulder, as compared to transaxially, which meant that the variation between ampoules themselves had a significant impact on the raw readings previously. The present analysis apparatus eliminates this source of variance by measuring a sample through the ampoule across multiple areas in a transaxial direction that are much more dimensionally accurate and consistent in the ampoule.

The present invention also simplifies calibration compared to using previous methods that require calibration against known standards with specific levels of light transmittance. Calibration of the present analysis apparatus may be done using an ampoule with either distilled or deionized water to provide values for light transmittance on all light paths without needing to rely on specially manufactured or known standards for different transmittance values.

In one aspect, a multi-transaxial spectrophotometric apparatus for analysis of biological samples comprises: a sample holder comprising a body defining a longitudinal axis; a longitudinally elongated container receptacle formed in the body, the receptacle configured to removably receive a sample container containing a biological sample fluid; a plurality of light sources longitudinally spaced apart along a first side of the receptacle; a plurality of light sensors longitudinal spaced apart along a second side of the receptacle; and a programmable microcontroller configured to control operation of the light sources and light sensors; wherein each light source is operable to transmit a light signal into the receptacle to illuminate the biological sample fluid when positioned in the receptacle, and each light sensor is operable to receive one or more light signals returned from the biological sample fluid. The first side may be opposite the second side.

In another aspect, a method for analyzing a biological sample fluid using a multi-transaxial spectrophotometer comprises: inserting a sample vessel containing a biological sample fluid into a container receptacle of the spectrophotometer which defines a longitudinal axis, the spectrophotometer including a plurality of light sources distributed along a first side of the receptacle at a plurality of elevations and a plurality of light sensors distributed along a second side of the receptacle at a plurality of elevations; selectively activating one or more of the light sources on the first side to transmit light signals into the receptacle which illuminates the sample fluid; selectively activating one or more of the light sensors on the second side to receive the light signals from the illuminated sample fluid; analyzing the light received by the light sensor using a programmable microcontroller configured to measure bacteria growth in the sample fluid; wherein the microcontroller is configured to control activating the light sources and light sensors. Each of the light sources are operable to transmit light signals into the receptacle in a direction which is transverse to the longitudinal axis of the spectrophotometer, and each of the light sensors are operable to receive light signals from the receptacle along a direction which is transverse to the longitudinal axis.

### BRIEF DESCRIPTION OF THE DRAWINGS

The present invention will become more fully understood from the detailed description and the accompanying drawings, wherein like elements are labeled similarly and in which:

FIG. 1 is a top perspective view of a biological sample analysis apparatus comprising one or more spectrophotometer units according to the present disclosure;

FIG. 2 is a bottom perspective view thereof;

FIG. 3 is a top exploded perspective view of;

FIG. 4 is a front view thereof;

FIG. 5 is a side cross sectional view thereof;

FIG. 6 is an enlarged detail taken from FIG. 5;

FIG. 7 is a top perspective view of the sample holder of the spectrophotometer unit;

FIG. 8 is a longitudinal cross-sectional view taken from FIG. 7;

FIG. 9 is a bottom perspective view of the sample holder;

FIG. 10 is a longitudinal cross-sectional view taken from FIG. 8;

FIG. 11 is a side view of the major side of the sample holder;

FIG. 12 is a longitudinal cross-sectional view of the sample holder in FIG. 11;

FIG. 13 is side view of a sealed sample vessel in the form of an ampoule usable with the sample holder and spectrophotometer units according to the present disclosure;

FIG. 14 is a longitudinal cross-sectional view taken from FIG. 11 showing the sample container receptacle, light transmission bores, and light reception bores of the sample holder;

FIG. 15 is a bottom view of the sample holder;

FIG. 16 is a transverse cross-sectional view of the sample holder taken from FIG. 11;

FIG. 17 is a top view of the sample holder;

FIG. 18 is a schematic diagram of the sample holder showing the light sources, light sensors, temperature control system, RFID sample tracking system components, and microcontroller which controls operation of the spectrophotometer units and sample testing; and

FIG. 19 is a high-level control block diagram of the microcontroller 200 showing features thereof and interface with external processors/devices.

All drawings are schematic and not necessarily to scale. Features shown numbered in certain figures which may appear un-numbered in other figures are the same features unless noted otherwise herein.

### DETAILED DESCRIPTION

The features and benefits of the invention are illustrated and described herein by reference to non-limiting exemplary (“example”) embodiments. This description of exemplary embodiments is intended to be read in connection with the accompanying drawings, which are to be considered part of the entire written description. Accordingly, the disclosure expressly should not be limited to such exemplary embodiments illustrating some possible non-limiting combination of features that may exist alone or in other combinations of features.

In the description of embodiments disclosed herein, any reference to direction or orientation is merely intended for convenience of description and is not intended in any way to limit the scope of the present invention. Relative terms such as “lower,” “upper,” “horizontal,” “vertical,”, “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise.

As used throughout, any ranges disclosed herein are used as shorthand for describing each and every value that is within the range. Any value within the range can be selected as the terminus of the range. In addition, any references to patents, patent application publications, or patent application numbers cited herein are hereby incorporated by reference in their entireties. In the event of a conflict in a definition in the present disclosure and that of a cited reference, the present disclosure controls.

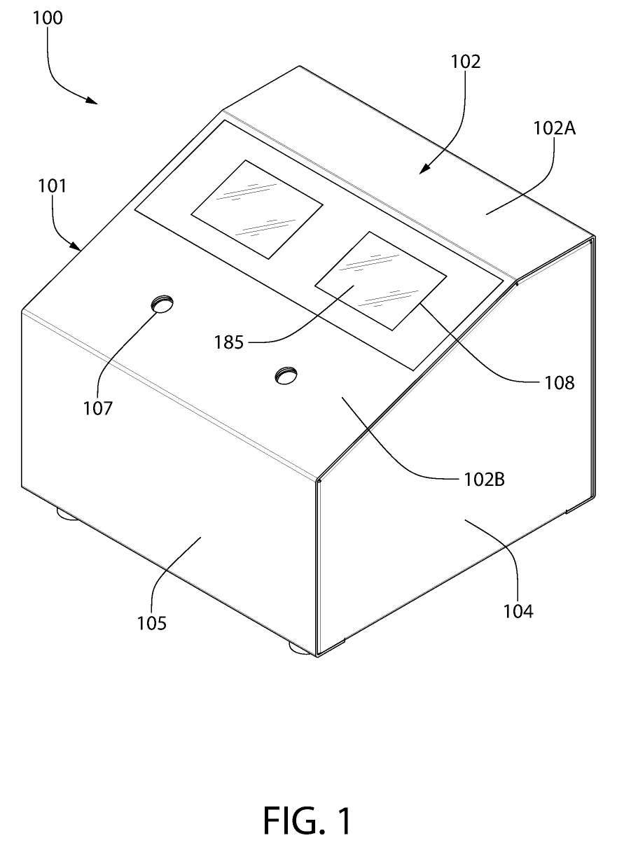

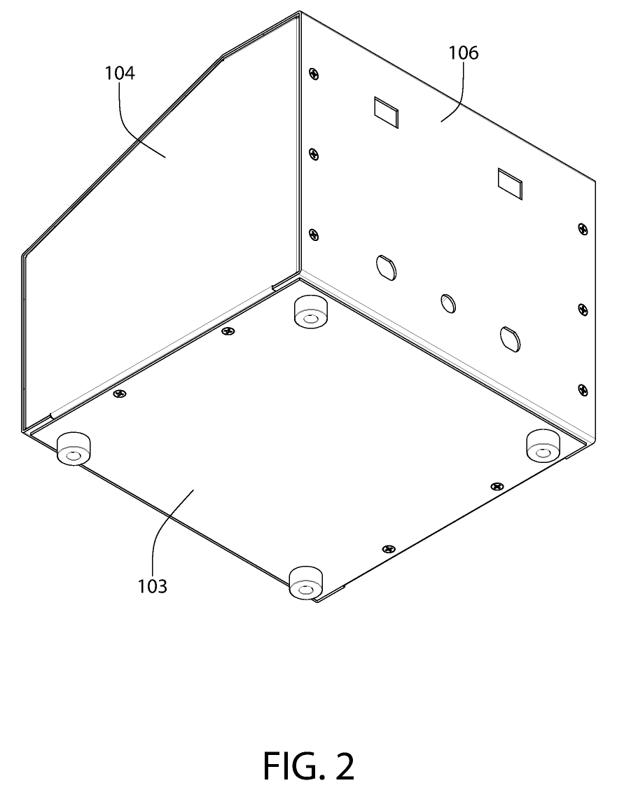

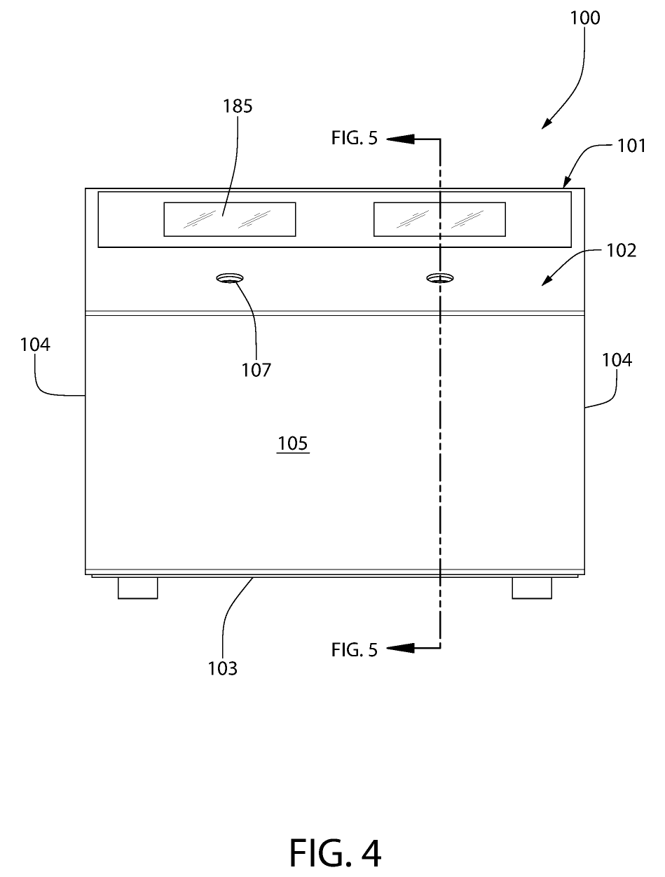

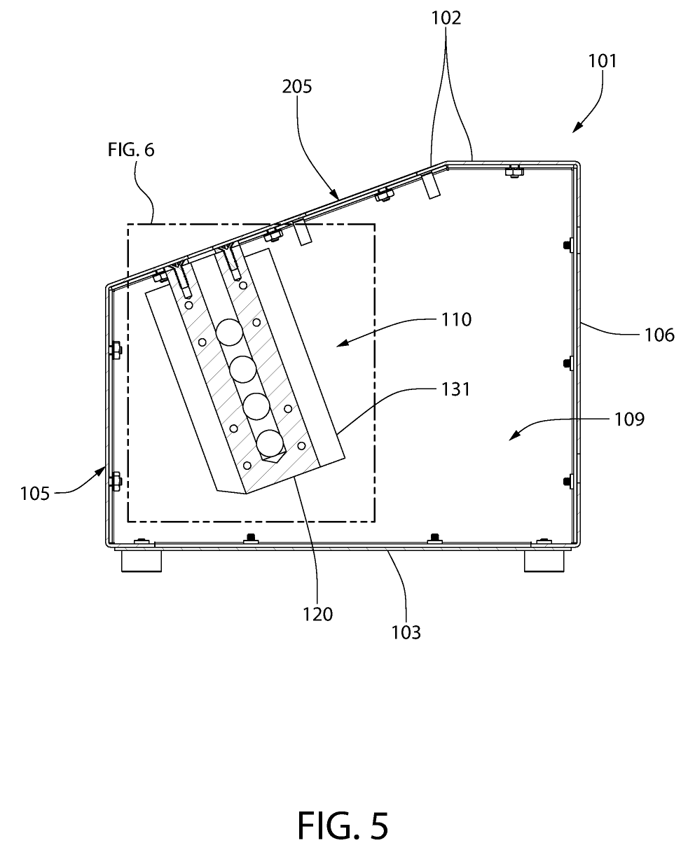

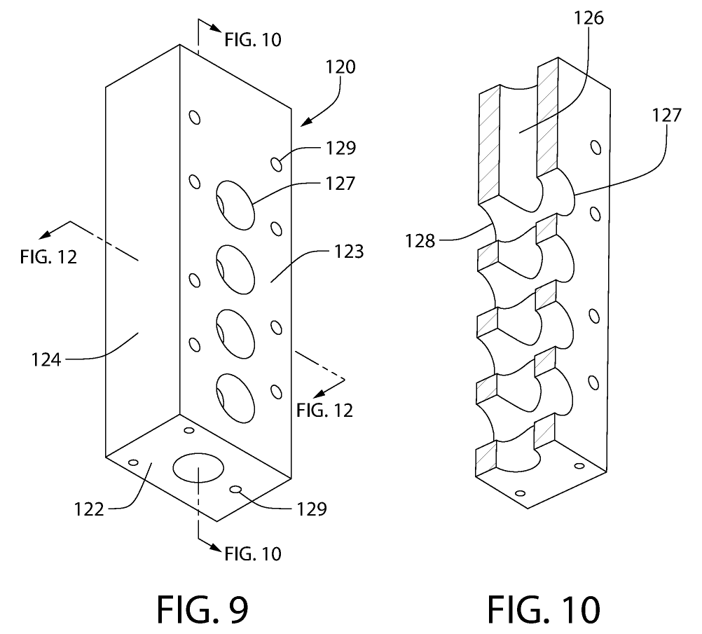

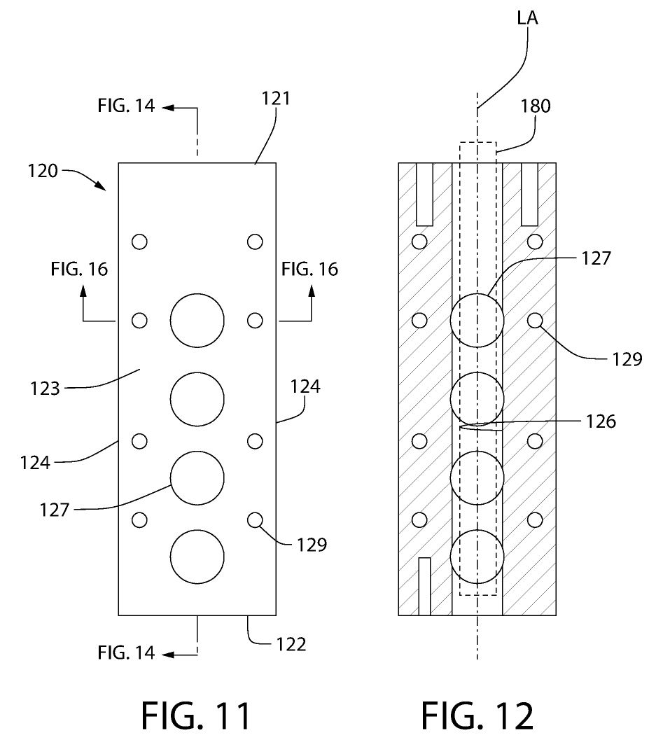

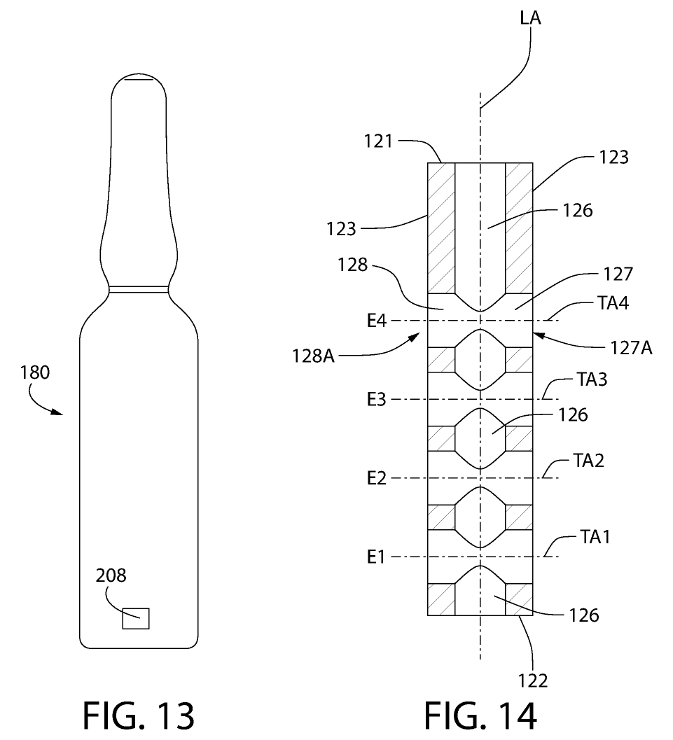

FIGS. 1-19 show various aspects and features of a biological fluid analysis system according to the present disclosure comprising an analysis apparatus 100 for analyzing a biological fluid sample for bacteria as described further herein. In one embodiment, the fluid sample may be urine and the analysis includes detection of a urinary tract infection (UTI).

Analysis apparatus 100 of the system generally includes a housing 101 supporting one or more spectrophotometer units 110 and a programmable microcontroller 200 configured to control operation of the units and analyze a biological sample fluid from a patient to measure biological cell mass and metabolism to determine the presence or absence of log phase bacterial growth in the fluid sample.

Housing 101 comprises a top wall 102, opposite bottom wall, front wall 105, opposite rear wall 106, and a pair of sidewalls 104 forming a complete enclosure. Each wall may be planar except the top wall may include a planar upper portion 102a and a planar sloped lower portion 102b disposed at an angle to the upper portion in one embodiment. Top wall 102 includes an entrance opening 107 for each spectrophotometer to insert a sample container 180 containing a biological sample fluid, and a window 108 through which an electronic display 185 of the spectrophotometer is visible to the user. The walls collectively define an internal cavity 109 configured to hold and support each spectrophotometer unit 110 provided and its associated microcontroller 200 which controls operation of each unit. Two spectrophotometer units 110 may be housed in apparatus housing 101 as shown; however, other embodiments may have more or a single unit. The housing may be formed of any suitable material including metal. Other materials, sizes, and shapes of housings may be used and does not limit the invention.

Spectrophotometry is a type of electromagnetic spectroscopy which enables quantitative measurement of the interaction of light with a substance such as a biological sample fluid as a function of wavelength. Measurement of light transmission or reflectance values of the fluid after being irradiated with light of one or more wavelengths is indicative of the properties of the fluid, and in the present case for example can ultimately be correlated to the presence or absence of log phase bacterial growth in the fluid sample for UTI detection. As further described herein, the present biological sample fluid analysis system utilizes at least two different wavelengths of light to detect a UTI condition.

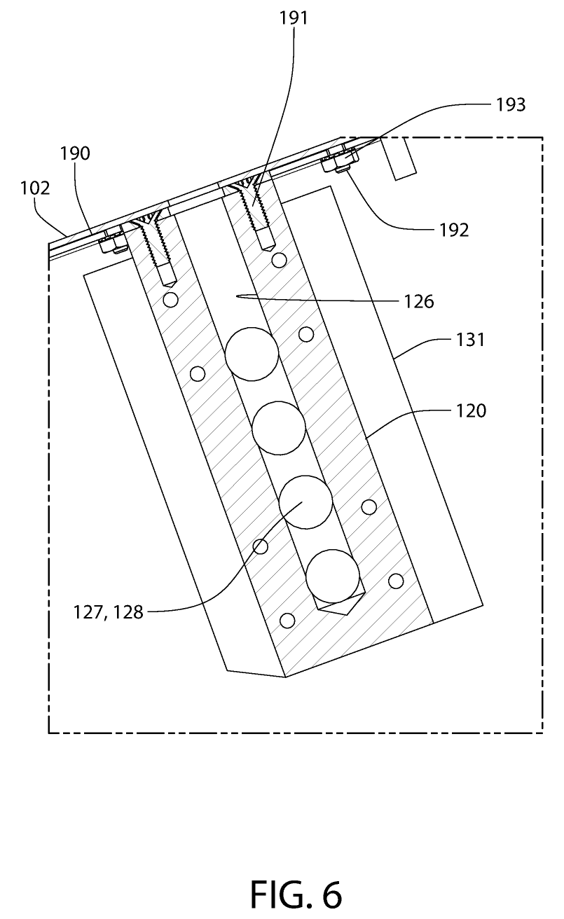

Each spectrophotometer unit 110 includes a sample holder 120 and an associated programmable microcontroller 200. The spectrophotometer unit in one embodiment may be supported in an overhead suspended manner from the top wall 102 of the apparatus housing 101. Mounting in this manner is convenient for aligning the container receptacle 126 of sample holder with its corresponding entrance opening 107 in the housing for inserting the sample container 180 containing the biological sample fluid for analysis into the spectrophotometer unit. To facilitate the suspended mounting, a mounting bracket 190 may be detachably coupled to the top 121 of sample holder 120 via threaded fasteners 191 inserted through corresponding threaded or unthreaded mounting holes 129 in the holder (see, e.g. FIG. 6). The bracket with sample holder attached in turn may be fastened to the inside surface of the top wall 102 of apparatus housing 101 which includes threaded studs 192. Nuts 193 coupled to the studs retain the mounting bracket 190 and in turn sample holder 120 to the housing in the foregoing suspended manner. Other means for coupling the sample holder to the housing may be used in other embodiments.

It bears noting that in other embodiments, the sample holder may be coupled to the apparatus housing 101 in other ways including non-suspended mounts. The manner of mounting the holder does not limit the invention.

Each sample holder 120 in one embodiment comprises an elongated body 120A defining a longitudinal axis LA which extends through the geometric centerline of the holder. The sample holder 120 provides a structure configured to removably retain the sample container 180 for spectrophotometric analysis and mounting the light sources, light sensors, and other appurtenances described herein. Sample holder 120 may therefore have a suitable configuration for these purposes. In one embodiment, the body 120A of the sample holder may be block shaped such as a rectangular cuboid as shown; however, other shaped bodies may be used as appropriate.

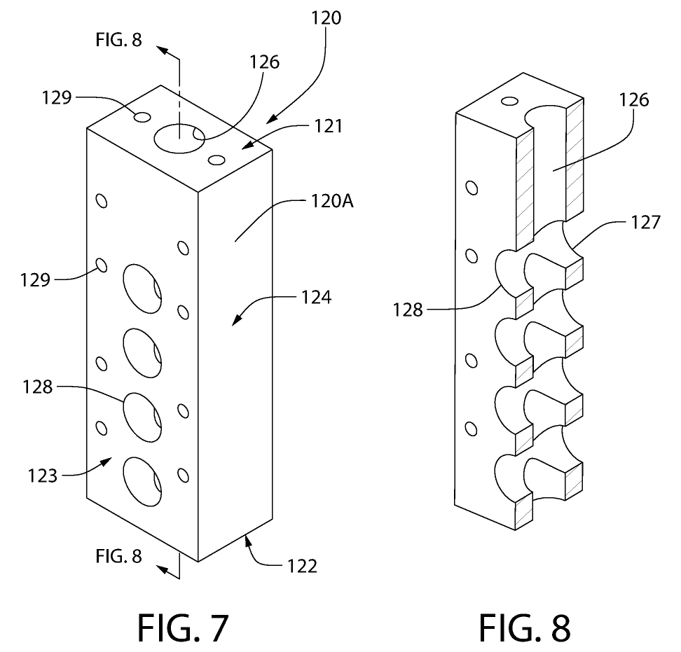

The sample holder body 120A includes a top 121, bottom 122, a first pair of opposing sides 123, and a second pair of opposing sides 124. All sides may be flat in one embodiment to facilitate mounting equipment thereto. Sides 123 may be major sides and sides 124 may be minor sides in one non-limiting embodiments as shown in FIG. 7 meaning that the major sides have a larger surface area than the minor sides. The larger major sides are convenient for mounting the electronic light boards 130 or 131 of the spectrophotometer unit 110 to the sample holder 120 as further described herein. The minor sides allow the holder body 120A to be more compact. In other possible embodiments, however, all sides may have equal surface area, and this aspect does not limit the invention.

The sample holder body 120A may have a monolithic structure in one embodiment being formed of a single piece or block of material. A suitable metallic or non-metallic material may be used for the holder body. In one embodiment, the body is formed of metal such as aluminum or another metal. Regardless of material type, using a rigid body is preferred to best support the light boards and suspended mounting of the sample holder 120.

Sample holder 120 further includes a longitudinally elongated container receptacle 126 formed in the body 120A which is configured to removably receive a sample container 180 containing a biological sample fluid for analysis. Receptacle 126 penetrates and extends from the top 121 of the sample holder to the bottom 122 along the longitudinal axis LA. The receptacle is preferably centered in the body from side to side and coaxial with the longitudinal axis which extends vertically through the receptacle as shown. In other possible embodiments, the receptacle might not be coaxial with longitudinal axis LA. In one embodiment, receptacle 126 may extend for the entire length of the sample holder from top to bottom and penetrate both the top and bottom surfaces defined by the top and bottom 121, 122. In other embodiments, it is possible that the receptacle may not penetrate the bottom of hold but may terminate close to the bottom defining a solid bottom surface.

The container receptacle 126 is complementary configured in shape and dimension to the shape and dimensions of the sample container 180 to accommodate the container therein with preferably minimal clearance on the sides just sufficient to slideably and removably receive the container. This prevents excess looseness and movement of the container once seated in the receptacle during the sample fluid analysis. In one embodiment, the receptacle 126 may have a circular cross-sectional shape forming a cylindrical cavity to match the sample container 180 which may concomitantly have an elongated cylindrical shape. In some embodiments, the sample container may be a cylindrical vessel such as without limitation a sealed ampoule. In one non-limiting embodiment, the ampoule may be made of borosilicate glass and is approximately 12 cm (centimeters) in length and 1.2 cm in diameter. Sample containers of other types and dimensions may be used in some embodiments such as for example without limitation capped test tubes or cuvettes with modifications to the wells.

Although a cylindrical sample vessel and matching container receptacle are described herein, other shapes of sample vessels and corresponding receptacles may be used.

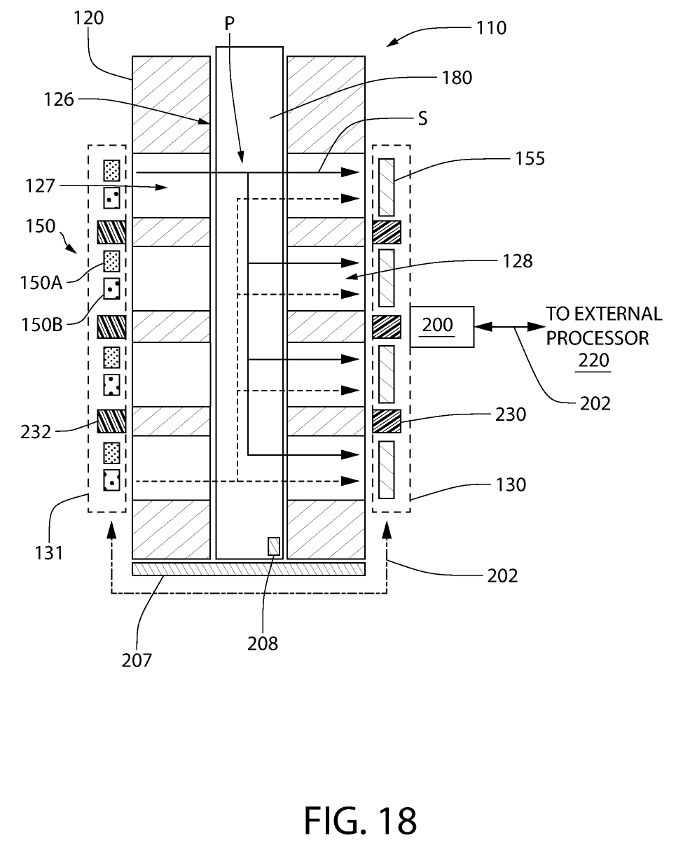

With particular reference to FIGS. 14 and 18 for the moment, sample holder 120 further comprises a plurality of light-transmission bores 128 each extending from one of the first major sides 123 to the container receptacle 126. The light-transmission bores each intersect and form a light pathway P to the receptacle from the first major side. Each light-transmission bore defines a light signal inlet port 128A configured to inject and transmit light from a light source into the receptacle. The light-transmission bores 127 are longitudinally spaced apart as shown along the height of the sample holder 120.

A plurality of light-reception bores 127 each extend from the opposite other major side 123 to the container receptacle 126. The light-reception bores each intersect and form a light pathway from the receptacle to the second major side. The light-reception bores each define a light signal outlet port 127A configured to receive light from the receptacle. The light-reception bores 127 are longitudinally spaced apart along the sample holder 120 as shown.

In the non-limiting illustrated embodiment, as shown, each light-reception bore 128 is concentrically aligned with a corresponding light-transmission bore 127 formed on the opposite side of the sample holder 120. Accordingly, a straight line of sight is formed from each light-transmission bore through the receptacle 126 to the light-reception bore. Although this is a preferred arrangement, it is possible that in other embodiments one or more of the light-reception bores might not be vertically located and arranged to concentrically line up with a corresponding light-transmission bore.

With particular reference to FIG. 14 in which the light-transmission bores are aligned with the light-reception bores, a light axis which extends transversely to the longitudinal axis LA of the sample holder 120 is defined for each pair of opposing light-transmission bores and light-reception bores. In one embodiment, four longitudinally spaced apart light-transmission bores and light-reception bores may be provided; however, more or fewer bores may be used in other embodiments when sufficient or necessary to obtain a full and accurate analysis of the biological sample fluid for the condition, substance, or organisms of interest.

Accordingly, the illustrated embodiment includes four transmission light axes TA1, TA2, TA3, and TA4 for the present arrangement which coincide with the locations of the light sources and light sensors of the sample holder 120. Each light axis corresponds to an elevation on the sample holder 120 designated as elevations E1, E2, E3, and E4. Each elevation has a corresponding pair of light-transmission and light-reception bores 127, 128 and one of the light transmission axes.

Light board 131 is a printed circuit board which holds a plurality of light sources 150. The light board is detachably coupled to one of the major sides 123 of sample holder 120 via any suitable means such as for example without limitation threaded fasteners for ease of installation and removal. Threaded or unthreaded fastener holes 129 may be provided in the sample holder for such coupling. At least one light source 150 is positioned at each light signal inlet port 128A defined by the light-transmission bores 128 when board 131 is mounted to the sample holder. Each light source emits and injects a light signal into the light signal inlet ports which in turn is transmitted to the container receptacle 126 of the sample holder 120 in order to illuminate the biological sample fluid F inside the sample container 180 when positioned in the receptacle.

Light emitting diodes (LEDs) of one or more suitable wavelengths may be used for the light sources in one embodiment. However, other types of light sources could be used in other embodiments and applications as appropriate.

In one embodiment, a plurality of light sources 150 are disposed at each light-transmission bore 127. And the plurality of light sources may include light sources of different wavelengths. In one embodiment, at least one light source 150A transmitting a light signal of a first wavelength and at least one light source 150B transmitting a light signal of a second wavelength (different than the first) may be disposed at the light signal inlet port 127A of each light-transmission bore 127. In one embodiment, a pair of light sources 150A and pair of light sources 150B may be disposed at each signal inlet port 127. Each light source 150A (e.g., LED) in one embodiment is slightly offset left/right from centerline of the container receptacle 126 (i.e. longitudinal axis LA) with the intention that if there was any misalignment the increase in the received signal from one side would offset a decrease in signal from the other side as detected by the light sensors 155. Also having a second of each LED is helpful for troubleshooting, diagnostics, and comparison purposes to make sure the spectrophotometer unit is functioning correctly. In addition, the dual LEDs provides more data for analysis of the biological sample fluid.

In one embodiment, light sources 150B may be infrared (IR) LEDs emitting light at a wavelength which may be in a range between about 780-1000 nm (nanometers). Light sources used in conjunction with the infrared light in one embodiment may be green LEDs emitting light at a wavelength which may be in a range between about 495–570 nm. These wavelengths are considered appropriate for UTI detection and may be for other conditions identifiable via spectrophotometric examination of biological fluid samples. In other embodiments depending on the infection being determined, a different combination of wavelengths or a single wavelength might be appropriate for use. Accordingly, the invention is not limited to use of the above specified wavelengths of the light sources which is just one example for UTI detection.

In one embodiment, the two green LEDs and two IR LEDs may be arranged in a square pattern, with the green LEDs at the same height as each other and laterally offset slightly left and right from the centerline of sample container 180 (e.g. ampoule) and the light axis, and the two IR LEDs at the same height as each other and offset slightly left and right of the sample container (see, e.g. FIG. 16).

The other light board 130 may be a printed circuit board which holds a plurality of light sensors 155 configured to sense and receive light from the sample holder container receptacle 126. The light board is detachably coupled to the opposite remaining major sides 123 of sample holder 120 via any suitable means such as for example without limitation threaded fasteners for ease of installation and removal. At least one light sensor 155 is positioned at each light signal outlet port 127A defined by the light-reception bores 127 when board 130 is mounted to the sample holder. In the non-limiting illustrated embodiment, a single light sensor may be used at each signal outlet port. Each light sensor is configured to sense and receive a light signal via the light signal outlet ports returned from the sample container 180 in the sample holder container receptacle 126 when the biological sample fluid F in the container is illuminated by the light sources. Accordingly, the light sensors 155 receive light after it passes through the sample fluid. Suitable photodetectors may be used for the light sensors 155. In one embodiment, the photodetectors used may be photodiodes.

It bears noting that in various embodiments the light boards 130 and/or 131 may be printed circuit boards containing integrated circuits including processors and microcontrollers.

In the present embodiment, light sensors 155 are placed at four different heights at each of the elevations E1-E4 along sample holder 120 from the bottom of the sample container. The light sensors are positioned at each light signal outlet port 127A to have a line of sight into the container receptacle 126 and sample container 180 (e.g. ampoule or other) when positioned therein.

In the non-limiting illustrated embodiment, the combination of four light sensors 155 and sixteen light sources 150 for the spectrophotometer 110 provides a total of 64 light paths for analysis which yields greater accuracy for detection of UTI conditions in a patient. This defines a quad transaxial spectrophotometer since light sources and sensors are provided in a transaxial arrangement perpendicular to the longitudinal axis LA of the sample holder and opposite each other at four different height or elevations along the sample holder 120. Each of the light sources are operable to transmit light signals into the receptacle in a direction which is transverse to the longitudinal axis, and each of the light sensors are operable to receive light signals from the receptacle along a direction which is transverse to the longitudinal axis LA of the sample holder 120.

### Sample Analysis Control System

The sample analysis control system in one embodiment includes a microcontroller 200 for and associated with each spectrophotometer unit 110 housed in the apparatus housing 101. Accordingly, each spectrophotometer unit has a separate and dedicated microcontroller which is programmed to control operation of the unit and allows the different units to function independently of each other for analysis of the sample. Each microcontroller is configured and operable to control operation of the including the lighting system (light transmission and detection), sample holder heating system to maintain the sample at a steady temperature, and other functions. In one embodiment, each microcontroller 200 may be located onboard one of the light boards 130 or 131 as shown in FIG. 18. The light boards and devices thereon communicate with the microcontroller regardless of which one of the boards on which it is mounted via wired and/or wireless communication links 202.

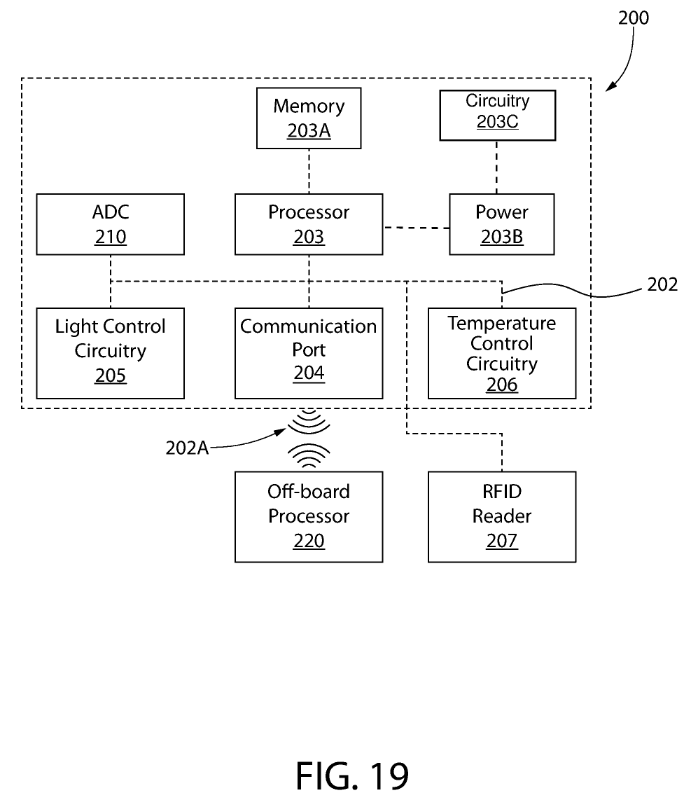

FIG. 19 is a high-level control block diagram showing some but not necessarily all of the features of each microcontroller 200 normally found in a microcontroller and other additional features relevant to use of the microcontroller in association with the spectrophotometer unit 110 and sample analysis disclosed herein. Each microcontroller is a self-contained computer on a chip mounted to one of the light boards 130 or 131 of each spectrophotometer unit 110 which includes all the usual electronic circuitry, devices, and appurtenances known in the art necessary to form a fully functional and programmable processor-based microcontroller unit (MCU). This may include for example one or more processors 203 (e.g. CPUs), memory 203A including non-transitory tangible computer readable medium which may store program instructions or code and data, programmable input/output peripherals including a communication port 204, power supply 203B, and all other necessary electronic appurtenances normally associated with a fully functional controller. These components are operably and communicably coupled together via wired or wireless communication links 202 configured to convey and exchange control signals and data between electronic devices and peripherals. Any suitable wireless protocols may be used for the communication links including WIFI, NFC (near field communication), Bluetooth, or others known in the art. The microcontroller of course includes the usual data and control buses (e.g. control bus, address bus, data bus) for data and control signal exchange between devices of the microcontroller. Microcontroller 200 can be configured via programming with appropriate program instructions to control the biological sample fluid analysis apparatus and associated processes described herein.

With continuing reference to FIGS. 18-19, light sources 150 and light sensors 155 are operably and communicably coupled to the microcontroller 200 via communication links 202 which are configured to carry control signals and data. The microcontroller 200 in one embodiment therefore includes light control circuitry 205 configured to control operation of both the light sources 150 and light sensors 155 including the sequencing and timing of initiating light transmission to illuminate the biological sample fluid and light detection by the sensors for spectrophotometric analysis of light returned from the fluid. The microcontroller can activate a series of one or more light sources and light sensors at different or all elevations on the sample holder 120 in any number of combinations to derive yield a multitude of light transmission measurements through the biological sample fluid which yields greater accuracy than conventional spectrophotometric detection methods.

The control system further includes an analog-digital converter (ADC) 210 used to monitor the amount of light reaching each of the light sensors 155 when one or more light sources 150 are turned on. ADC 210 may be integrated with the microcontroller 200 in some embodiments to convert the analog light signals received by the light sensors 155 into digital signals which are relayed to microcontroller processor 203. The ADC 210 is thus in operable communication with processor 203 via communication links 202. If not integrated directly into the microcontroller, ADC 210 may be located onboard either of the light boards 130 or 131 but preferably on the light board which contains the light sensors 155. In this case the digital light measurements are relayed to the microcontroller 200 which may be on the remaining opposite light board via communication links 202.

The microcontroller 200 may handle additional tasks associated with operation of the spectrophotometer unit 110 and analysis of light readings returned from the sample including handling a user interface, network communication with electronic devices external to the microcontroller, and others.

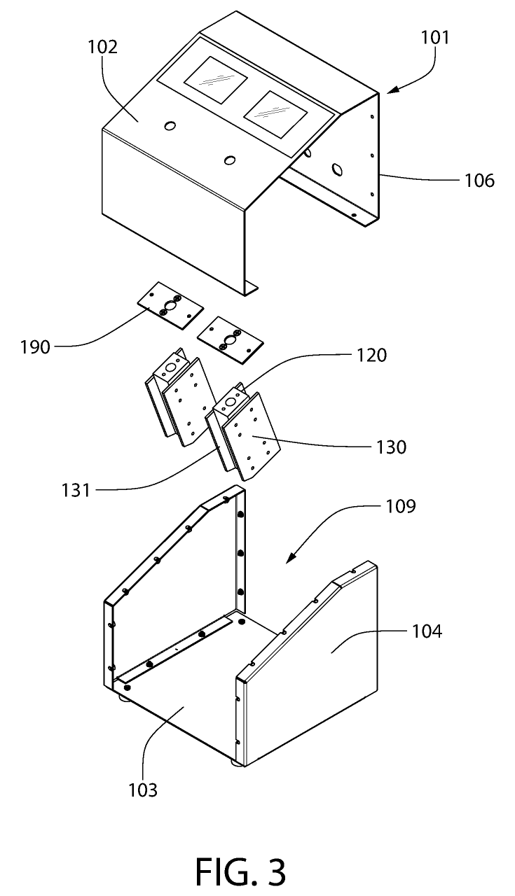

Microcontroller 200 is further configured and operable to receive the results of light transmission data received from the light sensors 155 (via communication with the ADC 210), and to record these results for further analysis such as comparing light measurements compiled at different times during the sample test period and determining the presence of bacteria growth in the biological sample fluid. In some embodiments, the microcontroller can be configured via programming to perform the sample analysis tasks beyond receiving and compiling light transmission measurements. In other embodiments as shown in FIG. 19, the analysis may be performed by a commercially available external electronic processing device 220 which may have more computing power to handle the detailed analysis of the light transmission data from the sample. Processing device 220 includes one or more central processing units (CPUs) and the usual ancillary electronic devices associated with a fully functioning computer of suitable type (e.g., laptop, desktop, tablet, server, etc.). Microcontroller 200 communicates with processor 220 via communication port 204 by a suitable wireless protocol 202A as illustrated, or wired means (e.g., control/data cables). In some embodiments as shown in FIG. 3 where two spectrophotometer units 110 are provided in the apparatus housing 101, the microcontroller 200 of each unit may communicate with the same external processing device which handles analysis of light transmission data and bacterial growth detection for both units.

During a typical UTI test, the microcontroller 200 in one non-limiting may cause the system to take a complete reading every 2 minutes for a period of 90 minutes. A complete reading will consist of the following: (1) Turning off power to all light sources and taking a reading from each light sensor to determine a baseline (ambient) light level; (2) Turning power on and off to each light source individually and taking a reading from each light sensor and subtracting the baseline light level to determine the effect of each individual light source on each individual light sensor; and (3) Recording all measured values for comparison over time throughout the test.

At the completion of a UTI test, an algorithm implemented by the external processing device 220 (or alternatively microcontroller 200 in some embodiments) determines the changes of the transmittance values in the green and IR light paths through the biological sample fluid from a plurality of different lighting scenarios implemented over the entire test period to determine if bacteria is growing in the sample which indicates the presence of a UTI infection.

Analysis apparatus 100 may optionally include a temperature control system under control of microcontroller 200 via its temperature control circuitry 206 as shown schematically in FIG. 18. The temperature control system includes a plurality of suitable commercially available electric resistance heating elements 230 in one embodiment. A suitable number of heating elements 230 are provided to evenly heat the entire sample holder body 120A from top to bottom. The heating elements are distributed in longitudinally spaced apart relationship along the height the sample holder 120. Heating elements 230 may be mounted on one of the light boards 130 or 131 on one of the major sides 123 of the holder. In other embodiments, the heating elements could be mounted internally in the container receptacle 126 of the sample holder 120 to directly heat the sample container 180. Each heating element 230 is in operable thermal contact with the side 123 of sample holder 120 which heats holder body 120A, and in turn the sample container 180 held in the container receptacle 126 to maintain the container at a preferred temperature of approximately 37 degrees C. (Celsius) to promote the growth of the bacteria in the biological sample fluid and to ensure a uniform environment for all tests performed. The stable temperature provided can also reduce any effects of temperature variation that may affect the light sources and light sensors, making the measurements more stable.

One or more suitable commercially available temperature sensors 232 are distributed in longitudinally spaced apart relationship along the height of sample holder 120 on the major side 123 opposite the side where the heating elements 230 are located. Temperature sensors 232 may be mounted to the remaining light board 130 or 131 which does not have heating elements. Each temperature sensor 232 is in operable thermal contact with the side 123 of sample holder 120 to monitor the temperature of the holder body 120A, which is indicative of the temperature inside the container receptacle 126. A suitable number of temperature sensors 232 are provided to obtain temperature readings which are indicative of the temperature of the entire sample holder body 120A from top to bottom. Any suitable commercially available type of temperature sensors may be used. In one embodiment, without limitation, thermistors may be used.

The microcontroller 200 via temperature control circuit 206 operates to control and regulate the heating elements 230 and receive temperature measurements from the temperature sensors 232 to maintain a preprogrammed setpoint temperature for the container receptacle (and biological sample fluid therein) which promotes the growth of bacteria in the sample if present. As noted above, the setpoint temperature in some embodiments may be about 37 degrees C (98.6 degrees Fahrenheit) which is generally considered average human body temperature.

According to another aspect of the invention, power control circuitry 203C is provided which is operably coupled to electric power source 203B and light control circuitry 205. Referring to FIG. 19, power control circuitry 203C is used to control the level of electric current supplied to each light source via light control circuitry 205 independently as necessary. In one embodiment, each light source can be turned completely off or be driven at one of a plurality of different levels of current which affects the intensity of light emitted by light sources 150 to illuminate the biological sample fluid. Biological sample fluids particularly for UTI detection (i.e. urine), may vary in opacity from patient to patient due to the presence of blood or puss in the urine. Accordingly, the sample fluid may vary from light and clear to dark and cloudy specimens. The ability to adjust the current level to each light source and intensity of light emitted advantageously allows the light signal intensity (i.e. dimmer or bright) to be varied to compensate for opacity variations in the sample fluid. The light sources 150 operably coupled to light control circuitry 205 may be driven at current levels adjusted based on the measured light transmittance values determined by the microcontroller 200 which may control the power control circuitry 203C in some embodiments, or the current level and concomitantly light intensity may be manually adjusted at the beginning of the test by visual observation of the sample by the user of the analysis apparatus 100. Such changes in light source driving current levels may be used as part of a light transmittance measurement by the microcontroller in some embodiments.

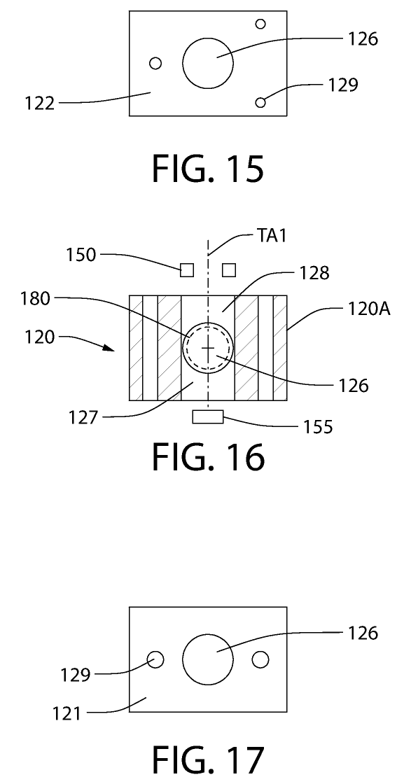

Each spectrophotometer unit 110 may also include an RFID reader 207 to track and correlate collected biological samples (fluid) with a specific patient from the point of sample collection through the sample analysis in the analysis apparatus 100 and control system. Referring to FIGS. 15 and 18-19, the RFID reader in one embodiment may be detachably mounted to the bottom of the sample holder 120 using mounting holes 129 provided and threaded fasteners. Other suitable coupling methods, however, may be used. In the illustrated embodiment, the flat plate-shaped body of RFID reader 207 may serve to close the bottom of the sample container receptacle 126 such that the container rests on the reader body when inserted in the sample holder 120 (see, e.g. FIG. 18). Each sample container 180 includes an RFID tag 208 located at a suitable position on the lower half of the container so that the tag is proximate enough to the reader 207 to be read when the container is inserted into the container receptacle. Each tag is uniquely tied to the specific patient.

In operation, the RFID tag 208 associated with a particular sample container 126 is automatically read by RFID reader 207 when the container is fully inserted into the sample holder 120. The container ID is relayed to the microcontroller 200 via wired or wireless communication links 202 so that the subsequent light transmittance measurements obtained by the spectrophotometer unit 110 from the biological sample fluid and results of the spectrophotometric analysis are properly tracked.

The present analysis apparatus 100 and associated microcontroller 200 enables numerous operational modes and aspects including the following.
A. Transmittance of light signals through the sample fluid can aggregated from all light sources 150 to all light sensors 155. This provides raw values of green and IR light transmittance throughout the sample container (e.g., ampoule) and biological sample fluid therein.
B. Transmittance from a single light source 150 to light sensor 155 at the same level or elevation. This provides raw values of green and IR light transmittance specifically across a short light path across the sample container 180 and sample fluid at different levels or elevations.
C. Transmittance from a single light source 150 to single light sensor 155 at a different (offset) vertical level or elevation. This provides raw values of green and IR transmittance specifically across longer light paths.

Cloudiness: By the microcontroller comparing the ratio of light transmittance directly across the sample container 180 (directly transverse light source and sensor in B above) to light transmittance at different levels/elevations along and across the sample container 126 (offset light source and sensor in C above), the degree of light scattering (cloudiness) of a sample can be determined. A clear sample will generally have a higher ratio of light transmittance directly across the sample container compared to light transmitted to an offset level. This allows the microcontroller 200 to adjust the electric current level to each light source and concomitantly light output level independently or collectively based on these light transmittance measurements by using the power control circuitry 203C described elsewhere herein.

Rate of change in transmittance between green and IR light paths: If turbidity or other phenomena are affecting raw light transmittance values, it would be expected that they would affect the IR and green transmittance values similarly. If the green and IR light transmittance values are varying at significantly different rates, it is indicative of a change in one or the other caused by the effects of either bacterial growth or bacterial metabolism.

Stratification and settling: By comparing the light transmittance at different levels/elevations of the sample container 180 over time, it can be determined if there is entrained material settling from the top of the container toward the bottom.

### Example Embodiments

1. A multi-transaxial spectrophotometric apparatus for analysis of biological samples comprising:
a sample holder comprising a body defining a longitudinal axis;
a longitudinally elongated container receptacle formed in the body, the receptacle configured to removably receive a sample container containing a biological sample fluid;
a plurality of light sources longitudinally spaced apart along a first side of the receptacle;
a plurality of light sensors longitudinally spaced apart along a second side of the receptacle; and
a programmable microcontroller configured to control operation of the light sources and light sensors;
wherein each light source is operable to transmit a light signal into the receptacle to illuminate the biological sample fluid when positioned in the receptacle, and each light sensor is operable to receive one or more light signals returned from the biological sample fluid.

2. The apparatus according to example 1, wherein the first side is opposite the second side.

3. The apparatus according to example 1, further comprising:
a plurality of light-transmission bores extending transversely from the first side to the receptacle to transmit light into the receptacle, at least one of the light sources positioned at each light-transmission bore;
a plurality of light-reception bores extending transversely from the second side to the receptacle to receive light from the receptacle, one of the light sensors positioned at each light-reception bore.

4. The apparatus according to example 3, wherein the light-transmission bores and light-reception bores are oriented perpendicularly to the longitudinal axis.

5. The apparatus according to example 4, wherein each light-transmission bore is arranged opposite a corresponding light-reception bore so that a straight line of sight is formed between pairs of light-transmission bores and light-reception bores at multiple elevations along the container receptacle.

6. The apparatus according to example 1, wherein the container receptacle is coaxially aligned with the longitudinal axis.

7. The apparatus according to example 1, wherein the container receptacle includes an open top end configured to removably insert the sample container therein.

8. The apparatus according to example 7, wherein the container receptacle has a circular cross-sectional shape, and the sample container is a sealed cylindrical vessel.

9. The apparatus according to example 1, wherein the sample holder is disposed inside a housing which supports the sample holder in a suspended manner from a top wall of the housing.

10. The apparatus according to example 2, wherein the microcontroller is configured to:
selectively activate one or more of the light sources to transmit the light signals into the biological sample fluid in the container receptacle; and
selectively activate one or more of the light sensors to receive the light signals returned from the sample fluid in the container receptacle.

11. The apparatus according to example 10, wherein the microcontroller is configured to:
activate a first light source of the light sources to transmit the light signal into the biological sample fluid in the receptacle; and
activate a first light sensor of the light sensors to receive the light signal returned from the sample fluid in the receptacle.

12. The apparatus according to example 11, wherein the first light source and the first light sensor are positioned opposite each other on the first and second sides respectively, the first light source and first light sensor being located at a same first elevation such that the light signal follows a linear light path through the biological sample fluid in the sample container between the first light source and the first light sensor.

13. The apparatus according to example 11, wherein the first light source is located at a first elevation on the sample holder, and the first light sensor is located at a different second elevation on the sample holder such that the light signal follows a non-linear light path through the biological sample fluid between the first light source and the first light sensor.

14. The apparatus according to example 11, wherein the microcontroller in a first operating mode is configured to inactivate all other light sources and light sensors except for the first light source and the first light sensor.

15. The apparatus according to example 10, wherein the microcontroller in a second operating mode is configured to activate all of the light sources and light sensors simultaneously.

16. The apparatus according to example 2, wherein each light source is a light-emitting diode.

17. The apparatus according to example 16, wherein a pair of light-emitting diodes of different wavelengths is disposed at each light-transmission bore.

18. The apparatus according to example 17, wherein each pair of light-emitting diodes comprises an infrared light-emitting diode and a green light-emitting diode.

19. The apparatus according to example 11, wherein the biological sample fluid is urine and the microcontroller is configured to analyze the light transmittance through the fluid for bacterial growth indicative of a urinary tract infection.

20. The apparatus according to example 19, wherein the microcontroller is configured and operable to measure biological cell mass and metabolism to determine the presence or absence of log phase bacterial growth in the biological sample fluid.

21. The apparatus according to example 20, wherein the microcontroller is configured to compile multiple light transmittance measurements from the sample fluid taken at different points in time during a test period by the microcontroller activating a plurality of light source and light sensor combinations, and compare the measurements for changes in light transmittance indicative of bacterial growth in the sample fluid.

22. The apparatus according to example 1, further comprising one or more heating elements controlled by the microcontroller that are configured and operable to heat the sample container to maintain a predetermined temperature.

23. The apparatus according to example 1, further comprising an RFID reader coupled to the sample holder, the RFID reader operable to read an RFID tag on the sample container when inserted into the receptacle which is correlated to a patient from whom the biological sample fluid was obtained.

24. The apparatus according to example 23, wherein the RFID reader is coupled to a bottom of the sample holder and closes a bottom end of the container receptacle.

25. The apparatus according to example 1, wherein each light source is paired with a corresponding opposing light sensor so that a straight line of sight is formed between pairs of light sources and sensors transversely to the longitudinal axis at three or more elevations along a height of the container receptacle.

26. The apparatus according to example 1, further comprising power control circuitry operably coupled to a power source and each of the light sources, the power control circuitry configured and operable to selectively adjust electric current to each light source at a plurality of current levels.

27. A method for analyzing a biological sample fluid using a multi-transaxial spectrophotometer, the method comprising:
inserting a sample vessel containing a biological sample fluid into a container receptacle of the spectrophotometer which defines a longitudinal axis, the spectrophotometer including a plurality of light sources distributed along a first side of the receptacle at a plurality of elevations and a plurality of light sensors distributed along a second side of the receptacle at a plurality of elevations;
selectively activating one or more of the light sources on the first side to transmit light signals into the receptacle which illuminates the sample fluid;
selectively activating one or more of the light sensors on the second side to receive the light signals from the illuminated sample fluid;
analyzing the light received by the light sensor using a programmable microcontroller configured to measure bacteria growth in the sample fluid;
wherein the microcontroller is configured to control activating the light sources and light sensors.

28. The method according to example 27, wherein each of the light sources are operable to transmit light signals into the receptacle in a direction which is transverse to the longitudinal axis of the spectrophotometer, and each of the light sensors are operable to receive light signals from the receptacle along a direction which is transverse to the longitudinal axis.

29. The method according to example 28, wherein the step of selectively activating one or more of the light sources includes activating a single first light source on the first side, and the step of selectively activating one or more of the light sensors includes activating a single first light sensor on the second side which receives the light signal from the illuminated sample fluid in the receptacle.

30. The method according to example 29, wherein the first light source and the first light sensor are located at a same elevation of the receptacle such that the light signal travels transversely to the longitudinal axis along a linear light path therebetween through the sample container.

31. The method according to example 29, wherein the first light source and the first light sensor are located at different elevations of the receptacle such that the light signal travels along a nonlinear light path therebetween through the sample container.

32. The method according to example 28, wherein each light source is a light-emitting diode.

33. The method according to example 32, wherein each elevation includes at least a pair of light-emitting diodes of different wavelengths.

34. The method according to example 33, wherein each pair of light-emitting diodes comprises an infrared light-emitting diode and a green light-emitting diode.

35. The method according to example 34, further comprising the microcontroller: conducting a preprogrammed testing sequence of turning on and off different combinations of light sources and light sensors over a test period of time; recording light transmittance measurements taken from the biological sample fluid during the sequence; and comparing the light transmittance measurements to identify growth of bacteria in the sample fluid.

36. The method according to example 35, wherein the biological sample fluid is urine and the microcontroller is operable to measure biological cell mass and metabolism to determine the presence or absence of log phase bacterial growth in the biological sample fluid which is indicative of a urinary tract infection.

37. The method according to example 27, wherein the first side of the receptacle is opposite the second side of the receptacle.

38. The method according to example 27, wherein the inserting step includes the microcontroller reading an RFID tag on the sample container when the sample container is inserted in the container receptacle.

39. The method according to example 27, further comprising the microcontroller activating one or more heating elements configured and operable to maintain the sample fluid at a preprogrammed setpoint temperature commensurate with human body temperature.

40. The method according to example 27, wherein each light source is paired with a corresponding opposing light sensor so that a straight line of sight is formed between pairs of light sources and sensors transversely to the longitudinal axis at three or more elevations along a height of the container receptacle.

41. The method according to example 27, further comprising power control circuitry operably coupled to a power source and each of the light sources, and further comprising a step of selectively adjusting electric current supplied to each light source from one of a plurality of current levels by using the power control circuitry.

While the foregoing description and drawings represent some example systems, it will be understood that various additions, modifications and substitutions may be made therein without departing from the spirit and scope and range of equivalents of the accompanying claims. In particular, it will be clear to those skilled in the art that the present invention may be embodied in other forms, structures, arrangements, proportions, sizes, and with other elements, materials, and components, without departing from the spirit or essential characteristics thereof. In addition, numerous variations in the methods/processes described herein may be made. One skilled in the art will further appreciate that the invention may be used with many modifications of structure, arrangement, proportions, sizes, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being defined by the appended claims and equivalents thereof, and not limited to the foregoing description or embodiments. Rather, the appended claims should be construed broadly, to include other variants and embodiments of the invention, which may be made by those skilled in the art without departing from the scope and range of equivalents of the invention.

What is claimed is:

1. A multi-transaxial spectrophotometric apparatus for analysis of biological samples comprising:
a sample holder comprising a body defining a longitudinal axis;
a longitudinally elongated container receptacle formed in the body, the receptacle configured to removably receive a sample container containing a biological sample fluid;
a plurality of light sources longitudinally spaced apart along a first side of the receptacle;
a plurality of light sensors longitudinally spaced apart along a second side of the receptacle; and
a programmable microcontroller configured to control operation of the light sources and light sensors;
wherein each light source is operable to transmit a light signal into the receptacle to illuminate the biological sample fluid when positioned in the receptacle, and each light sensor is operable to receive one or more light signals returned from the biological sample fluid.

2. The apparatus according to claim 1, wherein the first side is opposite the second side.

3. The apparatus according to claim 1, further comprising:
a plurality of light-transmission bores extending transversely from the first side to the receptacle to transmit light into the receptacle, at least one of the light sources positioned at each light-transmission bore;
a plurality of light-reception bores extending transversely from the second side to the receptacle to receive light from the receptacle, one of the light sensors positioned at each light-reception bore.

4. The apparatus according to claim 3, wherein the light-transmission bores and light-reception bores are oriented perpendicularly to the longitudinal axis.

5. The apparatus according to claim 4, wherein each light-transmission bore is arranged opposite a corresponding light-reception bore so that a straight line of sight is formed between pairs of light-transmission bores and light-reception bores at multiple elevations along the container receptacle.

6. The apparatus according to claim 1, wherein the container receptacle is coaxially aligned with the longitudinal axis.

7. The apparatus according to claim 1, wherein the container receptacle includes an open top end configured to removably insert the sample container therein.

8. The apparatus according to claim 7, wherein the container receptacle has a circular cross-sectional shape, and the sample container is a sealed cylindrical vessel.

9. The apparatus according to claim 1, wherein the sample holder is disposed inside a housing which supports the sample holder in a suspended manner from a top wall of the housing.

10. The apparatus according to claim 2, wherein the microcontroller is configured to:
selectively activate one or more of the light sources to transmit the light signals into the biological sample fluid in the container receptacle; and
selectively activate one or more of the light sensors to receive the light signals returned from the sample fluid in the container receptacle.

11. The apparatus according to claim 10, wherein the microcontroller is configured to:
activate a first light source of the light sources to transmit the light signal into the biological sample fluid in the receptacle; and
activate a first light sensor of the light sensors to receive the light signal returned from the sample fluid in the receptacle.

12. The apparatus according to claim 11, wherein the first light source and the first light sensor are positioned opposite each other on the first and second sides respectively, the first light source and first light sensor being located at a same first elevation such that the light signal follows a linear light path through the biological sample fluid in the sample container between the first light source and the first light sensor.

13. The apparatus according to claim 11, wherein the first light source is located at a first elevation on the sample holder, and the first light sensor is located at a different second elevation on the sample holder such that the light signal follows a non-linear light path through the biological sample fluid between the first light source and the first light sensor.

14. The apparatus according to claim 11, wherein the microcontroller in a first operating mode is configured to inactivate all other light sources and light sensors except for the first light source and the first light sensor.

15. The apparatus according to claim 10, wherein the microcontroller in a second operating mode is configured to activate all of the light sources and light sensors simultaneously.

16. The apparatus according to claim 2, wherein each light source is a light-emitting diode.

17. The apparatus according to claim 16, wherein a pair of light-emitting diodes of different wavelengths is disposed at each light-transmission bore.

18. The apparatus according to claim 17, wherein each pair of light-emitting diodes comprises an infrared light-emitting diode and a green light-emitting diode.

19. The apparatus according to claim 11, wherein the biological sample fluid is urine and the microcontroller is configured to analyze the light transmittance through the fluid for bacterial growth indicative of a urinary tract infection.

20. The apparatus according to claim 19, wherein the microcontroller is configured and operable to measure biological cell mass and metabolism to determine the presence or absence of log phase bacterial growth in the biological sample fluid.

21. The apparatus according to claim 20, wherein the microcontroller is configured to compile multiple light transmittance measurements from the sample fluid taken at different points in time during a test period by the microcontroller activating a plurality of light source and light sensor combinations, and compare the measurements for changes in light transmittance indicative of bacterial growth in the sample fluid.

22. The apparatus according to claim 1, further comprising one or more heating elements controlled by the microcontroller that are configured and operable to heat the sample container to maintain a predetermined temperature.

23. The apparatus according to claim 1, further comprising an RFID reader coupled to the sample holder, the RFID reader operable to read an RFID tag on the sample container when inserted into the receptacle which is correlated to a patient from whom the biological sample fluid was obtained.

24. The apparatus according to claim 23, wherein the RFID reader is coupled to a bottom of the sample holder and closes a bottom end of the container receptacle.

25. The apparatus according to claim 1, wherein each light source is paired with a corresponding opposing light sensor so that a straight line of sight is formed between pairs of light sources and sensors transversely to the longitudinal axis at three or more elevations along a height of the container receptacle.

26. The apparatus according to claim 1, further comprising power control circuitry operably coupled to a power source and each of the light sources, the power control circuitry configured and operable to selectively adjust electric current to each light source at a plurality of current levels.

27. A method for analyzing a biological sample fluid using a multi-transaxial spectrophotometer, the method comprising:
inserting a sample vessel containing a biological sample fluid into a container receptacle of the spectrophotometer which defines a longitudinal axis, the spectrophotometer including a plurality of light sources distributed along a first side of the receptacle at a plurality of elevations and a plurality of light sensors distributed along a second side of the receptacle at a plurality of elevations;
selectively activating one or more of the light sources on the first side to transmit light signals into the receptacle which illuminates the sample fluid;
selectively activating one or more of the light sensors on the second side to receive the light signals from the illuminated sample fluid;
analyzing the light received by the light sensor using a programmable microcontroller configured to measure bacteria growth in the sample fluid;
wherein the microcontroller is configured to control activating the light sources and light sensors.

28. The method according to claim 27, wherein each of the light sources are operable to transmit light signals into the receptacle in a direction which is transverse to the longitudinal axis of the spectrophotometer, and each of the light sensors are operable to receive light signals from the receptacle along a direction which is transverse to the longitudinal axis.

29. The method according to claim 28, wherein the step of selectively activating one or more of the light sources includes activating a single first light source on the first side, and the step of selectively activating one or more of the light sensors includes activating a single first light sensor on the second side which receives the light signal from the illuminated sample fluid in the receptacle.

30. The method according to claim 29, wherein the first light source and the first light sensor are located at a same elevation of the receptacle such that the light signal travels transversely to the longitudinal axis along a linear light path therebetween through the sample container.

31. The method according to claim 29, wherein the first light source and the first light sensor are located at different elevations of the receptacle such that the light signal travels along a nonlinear light path therebetween through the sample container.

32. The method according to claim 28, wherein each light source is a light-emitting diode.

33. The method according to claim 32, wherein each elevation includes at least a pair of light-emitting diodes of different wavelengths.

34. The method according to claim 33, wherein each pair of light-emitting diodes comprises an infrared light-emitting diode and a green light-emitting diode.

35. The method according to claim 34, further comprising the microcontroller: conducting a preprogrammed testing sequence of turning on and off different combinations of light sources and light sensors over a test period of time; recording light transmittance measurements taken from the biological sample fluid during the sequence; and comparing the light transmittance measurements to identify growth of bacteria in the sample fluid.

36. The method according to claim 35, wherein the biological sample fluid is urine and the microcontroller is operable to measure biological cell mass and metabolism to determine the presence or absence of log phase bacterial growth in the biological sample fluid which is indicative of a urinary tract infection.

37. The method according to claim 27, wherein the first side of the receptacle is opposite the second side of the receptacle.

38. The method according to claim 27, wherein the inserting step includes the microcontroller reading an RFID tag on the sample container when the sample container is inserted in the container receptacle.

39. The method according to claim 27, further comprising the microcontroller activating one or more heating elements configured and operable to maintain the sample fluid at a preprogrammed setpoint temperature commensurate with human body temperature.

40. The method according to claim 27, wherein each light source is paired with a corresponding opposing light sensor so that a straight line of sight is formed between pairs of light sources and sensors transversely to the longitudinal axis at three or more elevations along a height of the container receptacle.

41. The method according to claim 27, further comprising power control circuitry operably coupled to a power source and each of the light sources, and further comprising a step of selectively adjusting electric current supplied to each light source from one of a plurality of current levels by using the power control circuitry.
